(19) United States
(12) Patent Application Publication
(10) Pub. No.: US 2008/0051489 A1
(43) Pub. Date: Feb. 28, 2008

(54) Method for preparing a composition for treating flesh wounds; method of treating flesh wounds

(76) Inventor: Elia G. Dano, Oak Park, MI (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 11/511,125

(22) Filed: Aug. 28, 2006

(51) Int. Cl. A61K 8/72

(52) U.S. Cl. 523/105

(57) ABSTRACT

A method of repairing a flesh wound to, substantially, a pre-wound condition is provided. The method includes the steps of providing a composition prepared by a certain process and applying an amount of the composition to the wound sufficient to substantially cover the wound, wherein said application occurs about twice daily for a period of at least about ten days. The process for preparing the composition includes the steps of providing an ingredient consisting essentially of eggs; mixing the eggs until they form a substantially uniform mixture; adding the mixture to a cooking vessel; heating the cooking vessel until the mixture comprises a black liquid component; agitating the mixture during heating; collecting the black liquid component; and cooling the black liquid component. A method of treating a flesh wound is also provided, as well as a method of preparing a composition for treatment of a flesh wound.

### BACKGROUND

Many people have suffered and continue to suffer from flesh wounds of varying degrees. Such flesh wounds include but are not limited to first and second degree burns, abrasions, cuts, etc. Such flesh wounds can have a severe impact on a victim if they alter a victim's physical appearance, especially on a long term or a permanent basis.

For example, a sizeable second degree burn on a victim's face could be expected to have both physical and emotional effects on the victim. Depending on the nature and extent of the burn, the victim's face could take on a gruesome appearance. Such an appearance could put substantial strain on human interactions, both in a social context and in a work-environment context. This strain can lead to physical, emotional and financial consequences. These consequences can further challenge the victim's self-image. Worse, after the facial wound is physically healed, using conventional techniques, the flesh may never return to a normal, pre-burned condition in color or texture. Thus, the victim could be left to cope with long term, or even permanent, damage.

Unfortunately, flesh wounds are very common, and happen to people of all ages, races, gender, and socio-economic status. For example, children are burned in house fires, and soldiers suffer from attacks on the battleground.

Flesh wounds of varying severity are not limited to humans; mammalian skin generally is subject to flesh wounds. Family pets, for example, are also burned in house fires.

Thus, methods of treating flesh wounds are needed. A method of treatment that is capable of restoring wounded flesh to substantially a pre-wound condition is desired. A method of treatment that is inexpensive is desired. A method of treatment that is non-invasive is desired.

Similarly, methods of preparing a composition to treat flesh wounds are needed. A simple method of preparing a composition is desired. A method of preparing a composition that is relatively fast—within a few hours—is desired. A method of preparing a composition that is inexpensive is desired.

### SUMMARY

A method of treating a flesh wound is provided that has at least one of the desired traits of such methods. Similarly, a method of preparing a composition to treat flesh wounds is provided that has at least one of the desired traits of such methods.

A method of repairing a flesh wound to, substantially, a pre-wound condition is provided herein. The method comprises the steps of providing a composition prepared by a certain process and applying an amount of the composition to the wound sufficient to substantially cover the wound, wherein said application occurs about twice daily for a period of at least about ten days. The process of preparing the composition involves: (i) providing an ingredient consisting essentially of eggs; (ii) mixing the eggs until they form a substantially uniform mixture; (iii) adding the mixture to a cooking vessel; (iv) heating the cooking vessel until the mixture comprises a black liquid component; (v) agitating the mixture during heating; (vi) collecting the black liquid component; and (vii) cooling the black liquid component.

A method of preparing a composition to treat flesh wounds is prepared herein, as is the composition prepared by the method. The method of preparing a composition for treatment of a flesh wound includes the step of providing an ingredient consisting essentially of raw eggs. In one embodiment, no other ingredients are added to the eggs, whether natural or synthetic. In another embodiment, de minimus amounts of other ingredients, especially natural ingredients, are added. The method also includes the steps of mixing the eggs to form a mixture, then adding the mixture to a cooking vessel and heating the cooking vessel until the mixture comprises a black liquid component. The mixture can be agitated during heating. The black liquid component can be collected, then cooled and applied to a flesh wound.

### DETAILED DESCRIPTION

Surprisingly, it has been discovered that an all natural composition, prepared according to the methods disclosed herein and applied to flesh wounds according to the application methods disclosed herein, can repair flesh in a flesh wound to, substantially, a pre-wound condition.

Wounded flesh is “substantially” returned to a pre-wound condition if appears generally as it did in a pre-wound condition, as determined by visual inspection. A visual inspection can involve a naked eye comparison of wounded flesh that has been repaired using the methods disclosed herein with unwounded flesh, from a distance of at least about three feet in normal indoor lighting conditions. Normal indoor light conditions means, generally, subject to some natural and artificial light, but not necessarily subject to harsh lighting such as that often associated with fluorescent lights.

Repaired flesh passes a visual inspection even if the repaired flesh has minor inconsistencies in color and texture when compared to unwounded flesh. Inconsistencies in color and texture are minor if those inconsistencies are not observable by a visual inspection from a distance of at least about 15 feet in normal indoor lighting conditions.

An alternative method for determining if color of repaired flesh is substantially returned to a pre-wound condition is to compare a healed wound flesh sample with unwounded flesh using instrumentation such as a tri-stimulus colorimeter such as a Minolta ChromaMeter. Using such instrumentation, color is expressed as numerical coordinates, L*, a* and b*. The L* value expresses luminescence, reporting the relative brightness of the flesh. The a* value represents the balance between green and red. The b* value represents the balance between blue and yellow. If the a* value, the b* value and the L* value of the healed wound flesh color each differ no more than 15%, no more than 10%, or no more than 5% from the corresponding coordinate of the unwounded flesh, the wounded flesh is repaired, substantially, to a pre-wound condition.

An alternative method for determining if color of repaired flesh is substantially returned to a pre-wound condition is to compare a healed wound flesh sample with unwounded flesh using spectophotometry. Spectophotometry involves instrumentation that produces spectra showing wavelength regions where certain flesh colors are reflected. If the spectra of the healed wound flesh sample produces spectra that are substantially similar (peaks and valleys occurring generally within the same wavelength regions) to that of unwounded flesh, the wounded flesh is repaired, substantially, to a pre-wound condition.

### Preparing the Composition

Many embodiments of methods of preparing the composition that is used to repair flesh wounds are disclosed herein. In one embodiment, an all natural composition is prepared by cracking open one or more eggs, and placing resulting egg yolks and egg whites into a cooking vessel. Chicken eggs can be used, as can other types of eggs. Egg shells can be discarded. The cooking vessel may be any type of container capable of enduring heat. Non-limiting examples of suitable cooking vessels include covered and uncovered pots and pans.

The egg yolks and whites, together, may be subjected to agitation before, during and following heating of the cooking vessel. Agitation of any form can be used. Non-limiting examples of agitation include mixing and stirring. Heating may be accomplished using any suitable heating source or method. In one embodiment, a heating source comprises the application of low or medium or high heat supplied by a gas or electric range to the cooking vessel. In another embodiment, an oven is used as a heating source. In still another embodiment, an open fire is used to heat the cooking vessel. Generally, the higher the temperature applied to the cooking vessel, the faster the composition will be prepared.

After about 20 to 65 minutes (or after about 30 to 55 minutes) of heating in a covered vessel at a high temperature (as a non-limiting example, 450° F.-500° F.), the heated egg mixture can take on a brown color. Agitation and heating can continue until the egg mixture turns black, and generates a black liquid component. Any remaining solid egg substance in the cooking vessel may be discarded.

The black liquid component can be cooled using any known method. It can be allowed to set at about room temperature until it reaches room temperature. Alternatively, any form of refrigeration can be used to accelerate cooling to about room temperature. Cooling methods can be used that bring the black liquid component below room temperature, also. After the black liquid component is cooled, the cooled black liquid component is ready for use or storage.

### Optional Ingredients

In one embodiment, no additional ingredients are added to the composition, during or after the preparation of same. In another embodiment, the composition is substantially free of added ingredients, whether natural or synthetic. Substantially free means no more than about 1% by weight of any ingredient is added, where the weight of the entire composition is the 100% reference point. Without being bound by theory, it is believed that the nature of the composition is such that no preservatives are necessary.

Surprisingly, the composition has a shelf life of at least about five to seven years, without the addition of natural or synthetic preservatives. The composition need not be stored in reduced temperatures to maintain shelf life. Ideally, the composition is stored in a re-sealable container at temperatures ranging from about 10° C. to about 40° C., or alternatively from about 15° C. to about 25° C.

In another embodiment, additional natural ingredients (including but not limited to water or natural preservatives) are added in a de minimus amount to the composition (during or after preparation of same) such that the natural ingredients do not materially affect the basic and novel characteristics of the invention(s) of the appended claims. In one embodiment, the additional natural ingredients comprise, in total, no more than about 2.5% by weight of the entire weight of the composition. In another embodiment, the additional natural ingredients comprise, in total, no more than about 1.5% by weight of the weight of the composition. Suitable additional natural ingredients do not inflame or otherwise harm or aggravate flesh wounds.

Natural preservatives include but are not limited to oils and other extracts of Anise, Birch, Cajeput, Chinese anise, Chinese Cinnamon, Cinnamon, Clove, Cumin, Eucalyptus Fir, Pine, Garlic, Heliotrope, Juniper, Lavender, Lemon, Lemongrass, Meadowsweet, Melissa Balm, Neroli, Origanum, Orris, Parsley, Peppermint, Rose, Rose Geranium, Rosemary, Sassafras, Sweet Fennel, Sweet Orange, Thyme, Violet, Wild Thyme and Ylang Ylang and the like. Other natural ingredients could be added in de minimus amounts for aroma or rheology or stability or texture purposes, so long as said natural ingredients do not change the character or effectiveness of the composition.

In another embodiment, additional synthetic ingredients (including but limited to preservatives and fragrances and surfactants) are added in a de minimus amount to the composition (during or after preparation of same) such that the synthetic ingredients do not materially affect the basic and novel characteristics of the invention(s) of the appended claims. In one embodiment, the additional synthetic ingredients comprise, in total, no more than about 2.5% by weight of the weight of composition. In another embodiment, the additional synthetic ingredients comprise, in total, no more than about 1.5% by weight of the weight of the composition. Suitable additional synthetic ingredients do not inflame or otherwise harm or aggravate flesh wounds.

In another embodiment, additional synthetic and natural ingredients are added in a de minimus amount to the composition (during or after preparation of same) such that the ingredients do not materially affect the basic and novel characteristics of the invention(s) of the appended claims. In one embodiment, the total of the additional ingredients comprise, in total, no more than about 2.5% by weight of the weight of the composition. In another embodiment, the additional ingredients comprise, in total, no more than about 1.5% by weight of the composition. Suitable additional ingredients do not inflame or otherwise harm or aggravate flesh wounds.

TABLE 1
Provides a Guideline for Preparing the Composition

| Step | Instruction |
|---|---|
| 1 | Crack eggs and pour contents into a cooking vessel and mix |
| 2 | Heat the cooking vessel for about 30 minutes, stirring |
| | eggs periodically |
| 3 | When eggs turn black and take on the consistency of a liquid, |
| | remove liquid component from heat |
| 4 | Allow black liquid component to cool |

The guideline in Table 1 is not intended to limit the scope of the appended claims, but merely to teach one method of preparing a composition used to repair flesh wounds.

### Applying the Composition to Flesh Wounds

The composition prepared by the methods described can be applied to a flesh wound such as first and second degree burns, abrasions, cuts, etc.

### Initiating Treatment

Generally, it has been found that wounded flesh is more likely to be repairable to a substantially pre-wound condition if treatment begins shortly after an onset of a flesh wound. In one embodiment, a first application of the composition to the wound occurs within one day of the onset of the wound. In another embodiment, a first application of the composition to the wound occurs within two days of the onset. In another embodiment, a first application of the composition to the wound within a week of the onset.

### Application Methodology

Applying the composition to the flesh wound can occur in many ways. For each application, an amount of the composition is selected so that the composition can substantially cover the wounded flesh. The application may be done by hand or with an applicator of any kind. Non-limiting example of applicators include a brush or cotton swab or a dry or moistened towelette.

### Application Frequency

In one embodiment, the composition is applied to the wound at least once per day. The composition may also be applied to the wound at least twice daily, or at least about three times daily. More applications may be suitable.

### Application Duration

In one embodiment, treatment with the composition lasts for at least about seven days. Treatment period may vary, depending upon the nature and extent of the flesh wound. Treatment may occur over a period of at least about ten days, at least about fourteen days, or at least about twenty-one days, depending upon the circumstances. About half-way through the treatment period, harmed skin may peel off, and the appearance of regenerated skin may become apparent. At this point, the wound may still have areas discolored to a reddish hue. Applications of the composition can continue until the skin is back, substantially, to its pre-wound color.

Without being bound by theory, it is believed that the nature of the composition is such that no antibiotics are necessary for use with the composition.

TABLE 2
Provides A Guideline For Application of the Composition

| Step | Instruction |
|---|---|
| 1 | Identify a recently incurred flesh wound |
| 2 | Apply an amount of the composition to the wounded flesh |
| | twice daily using a small brush |
| 3 | If a portion of the wounded flesh dries out, re-apply the |
| | composition to at least the dried portion |
| | to keep the wounded flesh moisturized |
| 4 | Repeat applications of the composition during the period when |
| | wounded flesh peels or falls off and a new |
| | layer of skin appears |
| 5 | Continue applications of the composition to otherwise healed |
| | skin that remains red |
| 6 | After a period of applications lasting for about three weeks, the |
| | skin is substantially repaired to its pre-wound condition |

The guideline in Table 2 is not intended to limit the scope of the appended claims, but merely to teach one method of applying the composition to repair flesh wounds.

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. A method of preparing a composition for treatment of a flesh wound, comprising the steps of:
(a) providing an ingredient consisting essentially of raw eggs;
(b) mixing the eggs to form a mixture;
(c) adding the mixture to a cooking vessel;
(d) heating the cooking vessel until the mixture comprises a black liquid component;
(e) agitating the mixture during heating;
(f) collecting the black liquid component; and
(g) cooling the black liquid component.

2. The method of claim 1 wherein the composition is substantially free of added water.

3. The method of claim 1 wherein the composition is substantially free of added preservatives.

4. The method of claim 1 wherein the composition is substantially free of synthetic ingredients.

5. The method of claim 1 wherein the composition consists of one or more eggs.

6. The method of claim 1 wherein the agitating step comprises stirring.

7. The method of claim 1 wherein the heating step is carried out in a covered cooking vessel.

8. The method of claim 7 wherein the heating step is carried from about 25 minutes to about 35 minutes.

9. An all natural composition prepared by the method of claim 1.

10. A method of treating a flesh wound, comprising the steps of:
(a) providing an all natural composition prepared by:
(i) mixing eggs until they form a substantially uniform mixture;
(ii) adding the mixture to a cooking vessel;
(iii) heating the cooking vessel until the mixture comprises a black liquid component;
(iv) agitating the mixture during heating;
(v) collecting the black liquid component; and
(vi) cooling the black liquid component; and
(b) applying an amount of the all natural composition to the wound sufficient to substantially cover the wound, wherein said application occurs at least about once per day for a period of at least about seven days.

11. The method of claim 10 wherein the flesh wound is selected from the group consisting of a burn, an abrasion and a cut.

12. The method of claim 11 wherein the flesh wound is a first degree burn or a second degree burn.

13. The method of claim 10 wherein a first application of the all natural composition occurs within about one week of an on-set of the flesh wound.

14. The method of claim 13 wherein the first application of the all natural composition occurs within about two days of the onset of the flesh wound.

15. The method of claim 13 wherein the first application of the all natural composition occurs within about one day of the onset of the flesh wound

16. The method of claim 10 wherein the all natural composition is applied to the flesh wound twice daily for a period of at least about seven days.

17. The method of claim 16 wherein the applications of the all natural occur over a period of at least about fourteen days.

18. The method of claim 16 wherein the applications of the all natural occur over a period of at least about twenty-one days.

19. The method of claim 10 wherein the all natural composition is applied to the flesh wound about three times daily for a period of at least about seven days.

20. A method of repairing a flesh wound to, substantially, a pre-wound condition, comprising the steps of:
(a) providing a composition prepared by:
(i) providing an ingredient consisting essentially of eggs;
(ii) mixing the eggs until they form a substantially uniform mixture;
(iii) adding the mixture to a cooking vessel;
(iv) heating the cooking vessel until the mixture comprises a black liquid component;
(v) agitating the mixture during heating;
(vi) collecting the black liquid component; and
(vii) cooling the black liquid component; and
(b) applying an amount of the composition to the wound sufficient to substantially cover the wound, wherein said application occurs about twice daily for a period of at least about ten days.
